(19) United States
(12) Patent Application Publication
(10) Pub. No.: US 2011/0231336 A1
(43) Pub. Date: Sep. 22, 2011

(54) FORECASTING PRODUCT/SERVICE REALIZATION PROFILES

(75) Inventors: Jianying Hu, Bronx, NY (US); Aleksandra Mojsilovic, Yorktown Heights, NY (US)

(73) Assignee: INTERNATIONAL BUSINESS MACHINES CORPORATION, Armonk, NY (US)

(21) Appl. No.: 12/727,118

(22) Filed: Mar. 18, 2010

(51) Int. Cl. G06Q 10/00; G06F 15/18; G06N 5/02

(52) U.S. Cl. 705/348; 706/12; 706/54; 706/52

(57) ABSTRACT

Past realization profiles can be used to predict future realization profiles using a similarity rubric that emphasizes relationships between the past realization profiles. That similarity rubric might involve techniques including manifold characterization of past realization profiles; predictive modeling; and/or matrix factorization. Realization profiles might be related to business projects and track features such as ongoing resource expenditure, revenues realized, or percentage project completion. Realization profiles might relate to other applications such as effectiveness of medical treatment.

### BACKGROUND

The invention relates to the field of predicting a realization profile of a course of events.

Improving prediction of how certain events will be realized is relevant to many practical applications, such as business analytics, healthcare informatics, and medicine/biomedicine. One example in business application is the ability to predict how a certain project will be realized in the future—how it will bill revenue over time, how the cost will be spread, or how the resources will be consumed. Such information has implications to revenue management, resource management/deployment and business metrics. Another example in medical informatics is the ability to predict how certain actions/policies will influence medical outcomes, e.g. predicting diseases outcomes and trends, based on actions taken over a general population. In medicine/biomedicine, it is of interest to predict how a patient might respond to a particular treatment.

Forecasting has been discussed in numerous documents such as:
Armstrong, J. Scott (ed.) (2001) (in English). Principles of Forecasting: A Handbook for Researchers and Practitioners. Norwell, Mass.: Kluwer Academic Publishers. ISBN 0-7923-7930-6.
James D. Hamilton, Time-series Analysis, Princeton Univ. Press, 1994
Walter Enders, Applied Econometric Time Series, Wiley, 2009

This list is not complete, nor is it intended to imply that it would necessarily be obvious to combine any aspects of the various techniques disclosed in these documents.

### SUMMARY

Certain problems have been uncovered in the field of standard forecasting techniques. Although, predicting a realization of an event can be seen as a form of forecasting, the standard forecasting methodologies—e.g. time-series forecasting, or standard econometric methods discussed in the books listed above—cannot be applied to forecasting realization profiles, because the standard methods use previous data points to predict future realizations. Before a project starts, or a medication is administered—there are no past data points that could be used to predict the future. There are only limited basic characteristics of the event, e.g. project size, type and duration, in the case of predicting future project revenues and costs, or patient and medication information and characteristics in case of predicting the response to a treatment.

It would be desirable to develop tools and methodologies that would allow for prediction of realization profiles of an event, given the basic characteristics of the event. For example, if a service provider has just signed a new engagement to deliver network infrastructure services. The total value of the engagement to the provider is $250K, and the project is expected to last 6 months. For the purpose of business planning and budgeting, the services provider needs to estimate how this revenue will be realized, or how the cost will be incurred over the duration of engagement. Therefore the problem becomes, given the information about the new service engagement—total signed revenue, expected cost, duration, product/service group—predicting information such as how the engagement will bill throughout its duration, or how the cost will be spread, or how the resources will be utilized. In other words, can one predict its revenue profile, cost profile, and/or utilization profile?

In another example, given the basic information about the medication dosage—e.g. total amount administered, duration of the treatment, basic characteristics of a patient—the healthcare provider might be interested in predicting the response to treatment.

It would be desirable to develop a methodology that uses historical data on past events; and, for the new event, predicts its realization profile based on the characteristics of that event and its “similarity” to the past events.

Advantageously, a computer method includes running operations on at least one data processing device. The operations include:
maintaining at least one database of information embodied on a medium including historical realization profiles relating to a set of past courses of events;
creating at least one similarity rubric responsive to the historical realization profiles; and
deriving a predicted realization profile for a new course of events responsive to partial knowledge of a new instance and responsive to the similarity rubric.

A system includes:
at least one medium for embodying machine readable data and program code;
at least one interface for communicating externally;
at least one processor adapted to run operations responsive to the medium and interface, the operations include those from the method.

A computer program product is for performing operations. The computer program product includes a storage medium readable by a processing circuit and storing instructions run by the processing circuit for running a method. The method is the same as listed above.

Advantages, objects and embodiments will be further explored in the following discussion.

### BRIEF DESCRIPTION OF THE DRAWINGS

Embodiments will now be discussed by way of non-limiting example with reference to the following figures:

FIG. 1 shows a computer system on which an embodiment may be implemented.

FIG. 2 shows a flowchart of an embodiment of a method for forecasting a realization profile.

FIG. 3 shows a conceptual visualization of a manifold embodiment.

FIG. 4 shows an equation for deriving a manifold from a cloud of points.

FIG. 5 shows a flowchart for deriving a manifold embodiment.

FIG. 6 shows a flow chart for a predictive modeling embodiment.

FIG. 7 shows a flow chart for a matrix decomposition embodiment.

FIG. 8 shows a visualization of what a discretized manifold might look like.

FIG. 9 shows a visualization of what a projection of a fifteen dimensional manifold into three-space might look like.

### DETAILED DESCRIPTION

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable media may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

### DEFINITIONS

Examples of “realization” or a “realization profile” might include values of some criterion as a function of time. In the project management field, that criterion might include percentage of project completion, cost incurred, or revenue received. In the medical field that criterion might include some measure of treatment effectiveness.
The term “similarity rubric” as used herein will mean any computer implemented scheme responsive to a set of historical events that allows derivation of a projected realization profile responsive to partial information about a new instance and the scheme. Several examples of similarity rubrics are given herein. This includes a manifold embodiment, a predictive modeling embodiment, and a matrix decomposition embodiment. Those of ordinary skill in the art might devise other embodiments.

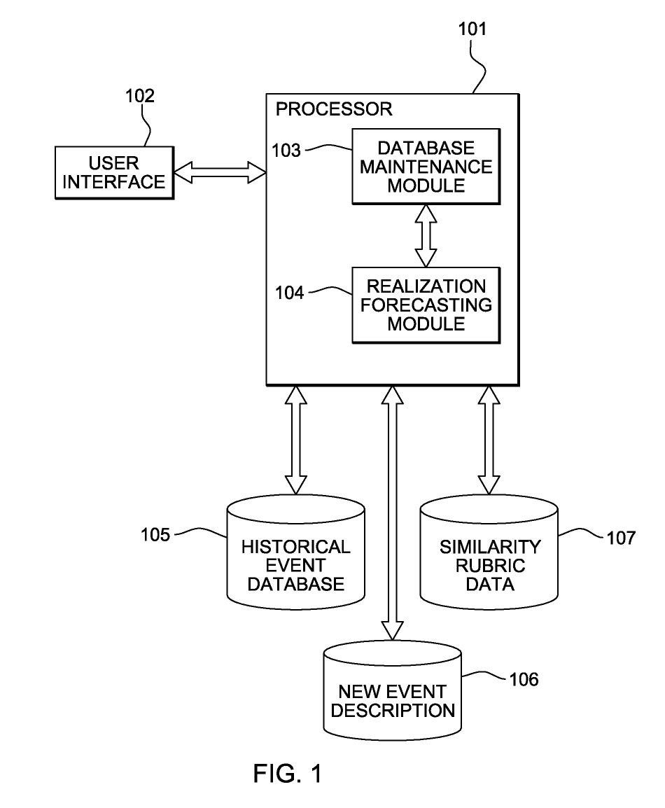

FIG. 1 shows a system including a processor 101 and a user interface 102. The processor includes a database maintenance module 103 and a realization forecasting module 104. At least one storage device embodies data and/or program code, including
machine readable database 105 including historical courses of events,
at least one new event starting description 106, and
data related to at least one similarity rubric 107.

Embodiments are not restricted to any particular type of processor, user interface or storage device. Data 105, 106, and 107 may be embodied in one or more storage devices. Individual data categories may be distributed over multiple storage devices.

The user interface 102, may include a display for giving information regarding expected project realization. Alternatively, the interface may give instructions to a user regarding how best to initiate a course of events.

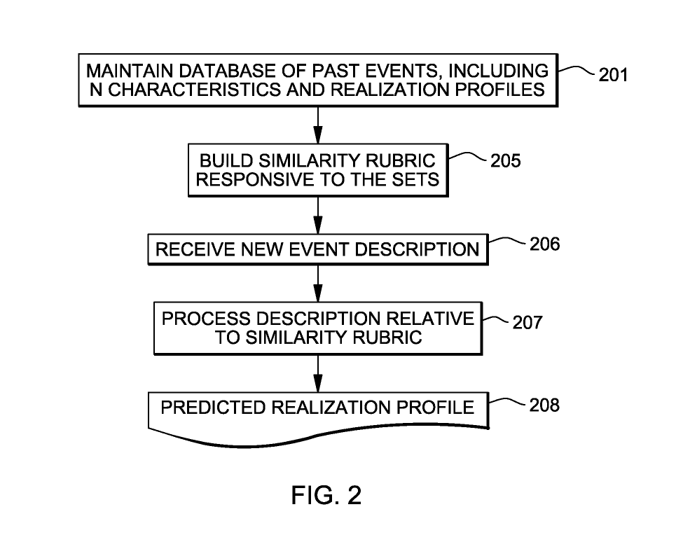

FIG. 2 shows a flowchart of a process or processes to be implemented on processor 101. At 201 a database 105 is maintained. This database includes past events in conjunction with N characteristics [c₁, c₂, . . . , cₙ] of such events and realization profiles of the events.

Then at 205 a similarity rubric 107 is constructed. When a new event comes in at 206, the new event 106 will be processed relative to the similarity rubric at 207. A projected realization profile will then be output at 208.

Embodiments of elements 205-208 will now be described.

Manifold Embodiment

The following symbols will be used:
Rn is an n-dimensional space of all possible realizations for the n measurements (features) associated with projects;
fx is a complete feature vector. The measurements/features might include duration, total revenue, project type, cost incurred, etc., which are known before the project starts
fx_known denotes the subset of features that are known before the project starts.
fx_unknown denotes the subset of features that are not known before the project starts. This subset includes timed samples of the realization profile, and in particular samples of revenue realization.
N is a number of historical projects
C represents a cloud of N points in Rn (i.e., all data points representing feature vectors of historical projects)
M represents a manifold—or shape—in the feature space derived from C
px represents a new project

The cloud C will form a manifold M, rather than being randomly distributed in space, when there are relationships between different features. For instance, one relationship might be that a long project might typically entail large revenue.

The problem of estimating the unknown realization profile for a new instance, px, can be then described as follows:
1. Use a set of N given points to learn the shape of manifold M. It would be desirable if M is smooth. This corresponds to 205.
2. Using the known set of features, fx_known(px), for the upcoming project px, determine the projection px_M of the new project onto the manifold M. The projection onto the manifold can be determined in many different ways. One example of finding the projection is finding a closest point on the manifold, with respect to the known set of features, and where the distance can be weighted/unweighted Euclidean or L1 distance, or any other distance metric. This corresponds to 206 and 207.
3. Read the entire feature vector for the projection, fx(px_M)=[fx_known(px_M) fx_unknown(px_M)], and use the remaining features fx_unknown(px_M) as the estimate for the unknown realization profile. This corresponds to 208.

A description of one way the manifold M can be constructed will follow

Intuitive description of forming a manifold from a cloud of points

Recovering a smooth manifold from a known set of points that belong to it can be seen intuitively as a problem of connecting neighboring points in n-dimensional feature space in a way that will create a smooth surface. The cloud of points C, seen as a subset of Rn, can be transformed into the manifold M. This can be achieved through an iterative process in which each point of C spreads itself in the direction of neighboring points. Little by little each point in C transforms itself into a short piece of curve oriented towards other points of C, and grows towards them. Eventually, all these pieces connect to one another so that the final curve is smooth and simply connected. At the same time, outliers are eliminated and the shape is regularized. This “spreading” process transforms C continuously into the smooth manifold M.

This process can be seen as a problem of constructing a smooth function u in Rn. The values of u are between 0 and 1. In particular, the value of u far from the manifold M is 1 and tends to 0 as the hypothetical observer approaches M. In a way, the function u represents the energy, E, of the spreading process—or the cost function of the spreading process—so that the final shape of M will correspond to the minimum of the energy. The system can start by assigning initial values of u to all points in Rn, and then update these values iteratively, by minimizing the selected cost function E.

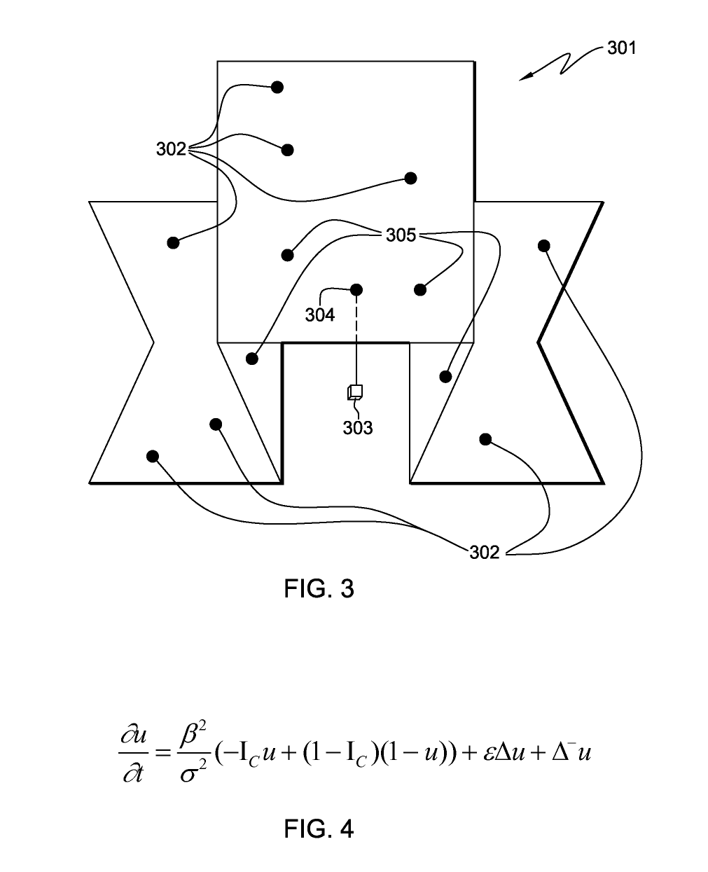

FIG. 3 is a simplified visualization of part of the process described above. In this simplified view, the manifold is a banner-shaped object 301 in three space. In other words, the space allows for only 3 characteristics to be represented. In a real problem, the space might have more or less dimensions. In a higher dimensional situation, the manifold might be much more complicated than this one. In a real problem, a set of related points might have more or less members than are illustrated in this figure. The manifold 301 approximates the configuration of a set of points 302, 305. These points may be on or near the manifold. When a new point 303 is to be added, it is projected onto the manifold at 304. Then a feature vector is derived based on the projection point 304. The derived feature vector may, for instance, contain snapshot values of the realization profile which will, as a result of the nature of the manifold, be interpolations between corresponding snapshot values of the realization profiles of historical courses of events.

Selecting the cost function E:

The cost function is typically a weighted sum of several energies, where each term contributes to favor or penalize a certain property of the desired shape. The achieved minimum will hopefully yield a satisfactory balance of all “competing” effects. The cost function will at least need two energy terms. Advantageously there will be a data attachment term, which will penalize shapes that contain a lot of points that do not belong to the initial cloud C. This prevents the spreading process from adding too many points and making M too “fat”. Symmetrically, shapes that do not contain all the points from C will advantageously be penalized as well. The second term is a regularization term, which favors better connected and smoother shapes. One way to obtain better connected shapes is to favor convexity. To summarize, the combined effect of all these terms will only favor spreading towards the neighboring points of C, because this is the only way the competing terms will reach an agreement.

This process corresponds to solving a partial differential equation (PDE) that performs iterative smoothing, i.e. anisotropic diffusion, on an implicit representation of the known set of points. The partial differential equation that corresponds to the above described construction of the cost function is set forth in FIG. 4. where,
u is a smooth function to be computed, corresponding to the functional representation of the desired manifold. As already mentioned, the value of u far from the manifold is 1, and tends to 0 as the hypothetical observer approaches manifold.
C is the initial cloud of points.
I is an indicator function of C, i.e. I_{C}(C)={1} and I_{C}(Rn/C)={1},
Δ⁻u is the negative part of the Laplacian of u, and
σ is the finest possible scale (determined by the resolution of the grid, e.g. for most common σ=1, two distinguishable points are two grid nodes.

The cost function is represented by different weights in the corresponding PDE, such as the parameters β and ε in the equation in FIG. 4. Parameters β and ε control the contribution of the different “forces” to the cost functions.

Details on how the cost equation is constructed and how to solve it can be found in J. Gomes, and A. Mojsilovic, “A variational approach to recovering a manifold from sample points”, Proc. European Conf. Computer Vision, ECCV 2002, Copenhagen, May 2002, the contents and disclosure of which are incorporated by reference as if fully set forth herein. Pursuant to the guidelines in the Gomes & Mojsilovic paper, values
β=ln(7+sqrt(48)) and
ε<<1
   may be derived to yield equal contributions. Those of ordinary skill in the art might derive other values for these parameters as a matter of design choice, if other criteria are adopted.

Manifold Computation Operations

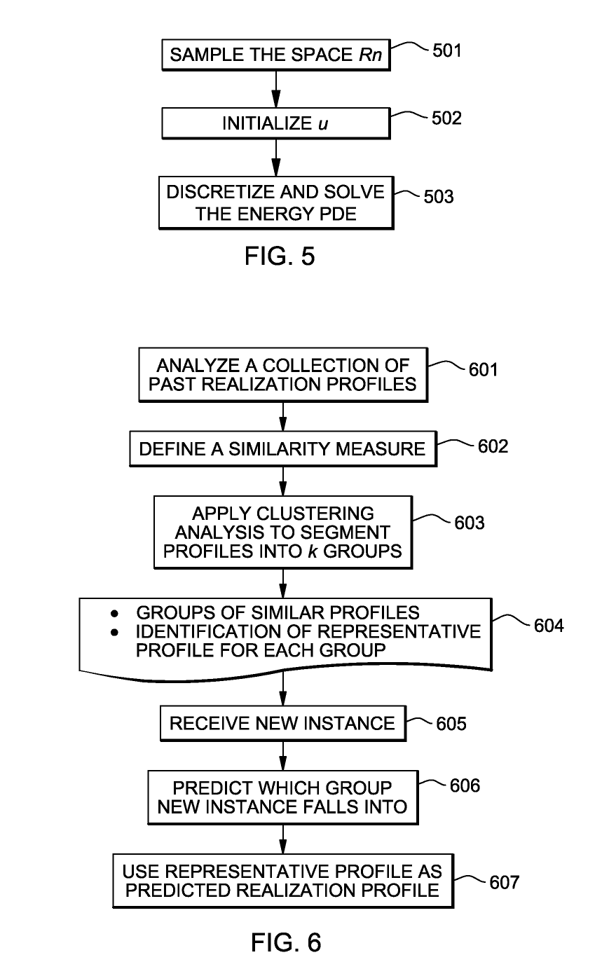

FIG. 5 shows operations in a manifold computation operation. These include:
1. At 501, sample the space Rn. (Note that the sampling can be uniform if the space is Euclidean, but may not be).
2. At 502, initialize u, the system can simply initialize u as follows: uo(C)={0} and uo(Rn/C)={1}. Another way that is more robust to outliers and behaves better is suggested in the J. Gomes article listed above.

3. At 503, discretize and solve the energy PDE. One way to do this is to apply standard explicit forward scheme for the time derivatives and the standard explicit centered schemes for the spatial derivatives.

Predictive Modeling Embodiment

FIG. 6 shows a flowchart of a predictive modeling embodiment.

The predictive modeling approach may start at 601 by analyzing a collection of past realization profiles, each realization profile being represented by a sequence of features (e.g., % of revenue realized at given time points after item start). One assumption may be that the collection of past realization profiles form a good representation of all possible realization profiles. A similarity measure can then be defined at 602 to determine how similar any two realization profiles are to each other. One such similarity measure could be Euclidean distance, after each sequence is normalized to a predetermined length D. Another similarity measure could be based on dynamic time warping which does not require normalization of sequence lengths. Dynamic time warping is described, for instance, in C. S. Myers and L. R. Rabiner, “A Level Building Dynamic Time Warping Algorithm for Connected Word Recognition”, IEEE TRANSACTIONS ON ACOUSTICS, SPEECH, AND SIGNAL PROCESSING, VOL. ASSP-29, NO. 2, APRIL 1981. The contents and disclosure of which are incorporated by reference as if fully set forth herein.

Once a similarity measure is defined, clustering analysis can segment revenue profiles into k groups at 603. Many standard clustering techniques can be used for this purpose, including hierarchical clustering and k-means type of clustering, both of which are described in A. K. Jain, M. N. Murthy and P. J. Fynn, “Data Clustering: A Review”, ACM Computing Reviews, November 1999; or a combination of both per B. Chen, P. C. Tai, R. Harrison, and Y. Pan, “Novel Hybrid Hierarchical-K-Means Clustering Method (H-K-Means) for Microarray Analysis,” Proceedings of the 2005 IEEE Computational Systems Bioinformatics Conference Workshops (BCSBW'05), Stanford, Calif., 2005. The clustering analysis process segments all realization profiles into groups of similar profiles, and identifies one most “representative” profile to represent each group per 604. The problem of realization profile prediction can then be formulated as: given the known attributes of a new instance at 605, it is desirable at 606 to predict which group of realization profiles the instance will likely have, and then use the representative realization profile of the identified group as the predicted realization profile per 607.

More formally: given the m known features—e.g., projected total revenue, project type, cost incurred, projected total duration, etc.—of an item p, it is desired to predict which one of the k realization profile groups, derived from the clustering analysis described above, it will most likely belong to. This can be viewed as a classic multi-class classification problem; and many standard methods, including regression analysis, classification and regression trees (CART), neural networks, and Support Vector Machines can be used to solve the problem. Examples of these may be found in Thomas Mitchell, Machine Learning, McGraw-Hill, 1997

Non-Negative Matrix Factorization

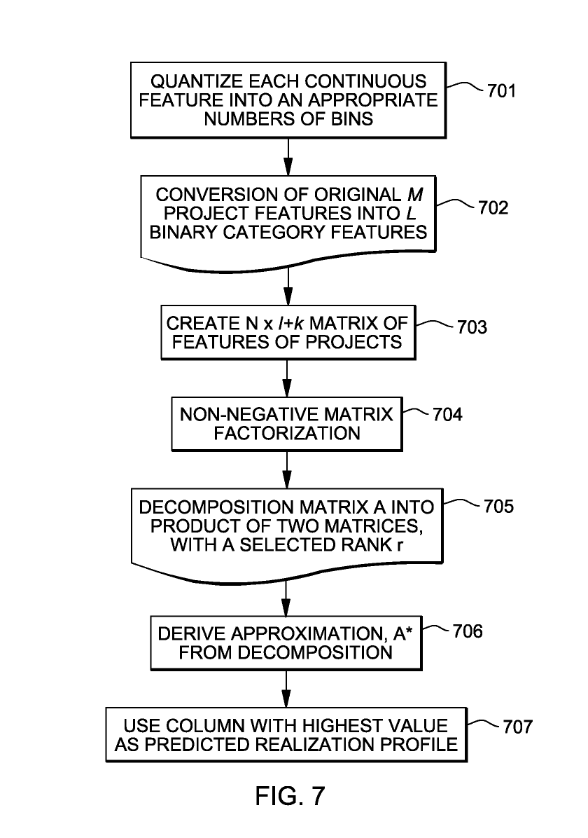

One new predictive modeling approach that has been found to be successful in recommendation systems is non-negative matrix factorization—per Y. Koren, R. Bell and C. Volinsky. “Matrix Factorization Techniques for Recommender Systems”, IEEE Computer, volume 42 (8), 2009—can also be applied here. One embodiment of such an approach is shown in FIG. 7. At 701, each continuous feature is quantized into an appropriate number of bins using standard quantization techniques such as those disclosed in A. Gersho and R. M. Gray, Vector Quantization and Signal Compression, Springer, 1991. By doing this, the original m project features (e.g., projected total revenue, project type, projected total duration, etc.) are converted into l binary “category features”, with each binary category feature representing one bin of one of the original features per 702. At 703, a matrix A is constructed with n projects (including past projects with group assignments, and new projects to be assigned) by (l+k) features. Each row of A represents a project. Each one of the first l columns represents a binary feature, and each one of the last k columns represents the k realization profile groups. Initially, the last k columns of each row representing a new project is 0, because their realization profile group is unknown. For each row representing a past project, one and only one of the last k columns will be 1—representing the group it has been assigned to. A non-negative matrix factorization method can then be used to decompose matrix A into a product of two matrices, with a selected rank r per 705. The system can then obtain an approximate version of A, called A*, per 706, which now has non-zero entries for each new project in the last k columns. The column corresponding to the highest value in matrix A* is then selected as the predicted revenue group for the new project at 707. The intuition here is that the low rank imposed in the decomposition process helps to “discover” any underlying relationships between the binary category features—known to all projects—and the revenue profile group ids, which are known only to past projects. These relationships are then exploited in the reconstruction process to help predict the group of the new projects.

### SOME EXAMPLES

To better illustrate the operation of the invention, two simplified examples will now be presented. These examples are presented with few dimensions, so that they can be more easily illustrated. It should be understood that, in practice, examples might well have many more dimensions and the clouds of points would then be modeled as existing in hyperspace.

### Example 1

Hypothetically, it will be assumed that a service provider company X has signed up a new client to deliver an engagement of type Y. The expected cost of the engagement is $100K. For purpose of financial planning, budgeting and resource planning, the provider is interested in estimating how the $100K will be realized over the upcoming two quarters Q1 and Q2.

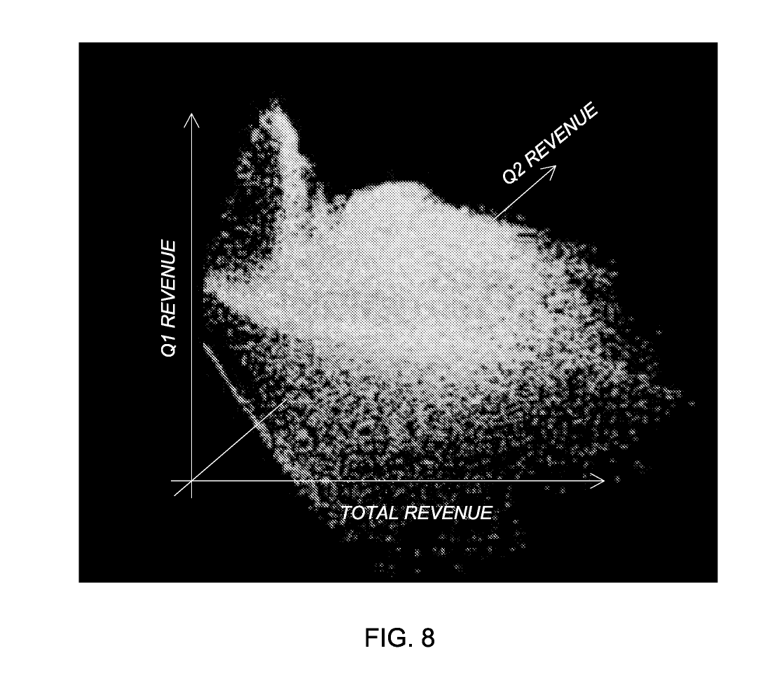

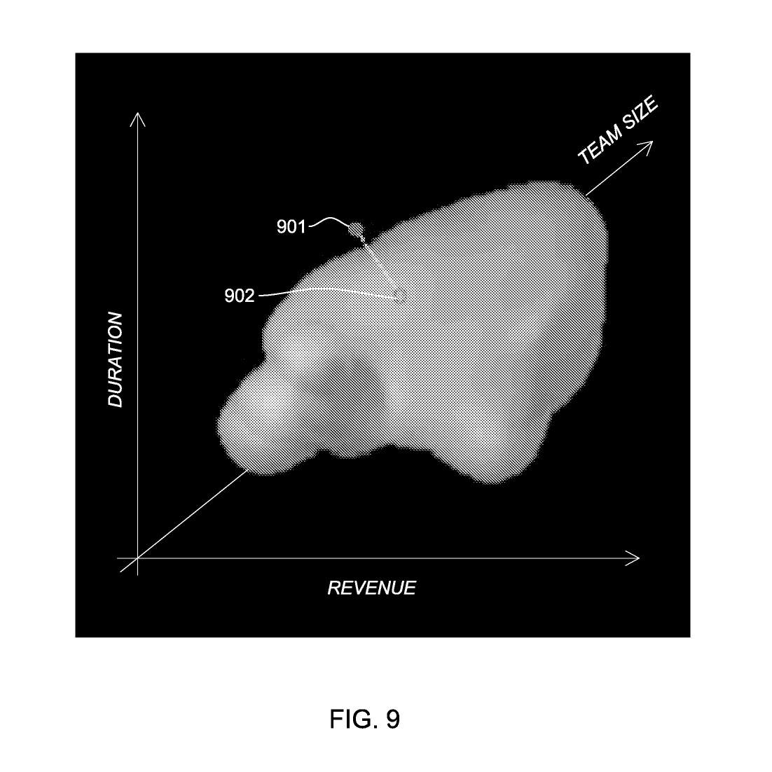

In order to do so, the system would aggregate the information on all prior engagements of type Y. This will include their total signing revenue, and the revenue realized in the two quarters following the engagement. Based on the collected historical information, the system would build a manifold in three-dimensional space. FIG. 8 is a conceptual diagram illustrating what a discretized view of the manifold might look like. Ideally a manifold is a surface; however in computer applications, that manifold will typically be represented as a set of points sampled from the surface, due to the digital representation. These points look like a cloud of points, but are not the original data points. They are a discretized manifold. The sampling can be coarse as in FIG. 8, or fine, where it would appear smoother as in FIG. 9. The sampling density for the manifold—or the finest resolution—will depend on the type of application. The dimensions in the 3D space correspond to revenue, amount billed in the first quarter and amount billed in the second quarter following the engagement. If one wanted to be able to predict more quarters, or more details within a quarter, more dimensions would have to be added.

To estimate the amounts service provider X will bill for the new project in the following two quarters, the system can take the known information for the new project (i.e. in the present simple example, signing revenue of $100K) and project that point onto the manifold. In this simple example, that corresponds to the exhaustive search of all points on the manifold to find the one that is closest to $100K revenue. Once the closest point has been identified, the system can read out the other two dimensions as an estimate for the revenue amounts that will be realized. For example, the system might derive, using this process, a forecast that the given project will bill $10K in the first quarter and $90K in the second. If more than one point on the manifold are equally close, then the average of these points is taken as the predicted profile.

### Example 2

FIG. 9 shows a second simple example. A service provider company X has signed up a new client to deliver an engagement of type Y. The expected amount of the engagement is $1.5M, duration is seven months and the size of the delivery team is 22 people. For purpose of financial planning, budgeting and resource planning, the provider is interested in estimating how the $1.5K will be realized over the duration of the engagement.

In order to do so, the system might aggregate the information on all prior engagements of type Y. This would include their total signing revenue, their duration, team size, and the revenue realization profiles for the entire year following the engagement. Based on the collected historical information the system can build a representation of a manifold in 15-dimensional space. The dimensions correspond to the following information:
d1—revenue,
d2—duration,
d3—team size, and
d4 to d15 corresponding to revenues billed in every month of the year following the engagement.

To estimate the amounts Provider X will bill for the new project over the following year, the system can take the known information for the new project—i.e., in this example, signing revenue of $1.5M, duration of 7 months and team size of 22—and project that point onto the manifold. FIG. 9 shows representation of the projection of the manifold in the sub-space of the three known dimensions, namely: revenue, duration, and team size.

The point 901 outside the manifold represents the new project.

Once the closest point 902 on the manifold has been identified—using only the three known dimensions—the implementing system can read out the other 12 dimensions as an estimate for the revenue amounts that will be realized. For example, the system might derive from this process a forecast to the effect that the given project will realize its $1.5M as 100K, 200K, 200K, 200K, 200K, 200K, 100K, 100K, 50K, 50K, 50K, 50K.

If more than one point on the manifold is equally close to the projection 902, then the average of these points is taken as the predicted profile.

Although the embodiments of the present invention have been described in detail, it should be understood that various changes and substitutions can be made therein without departing from spirit and scope of the inventions as defined by the appended claims. Variations described for the present invention can be realized in any combination desirable for each particular application. Thus particular limitations, and/or embodiment enhancements described herein, which may have particular advantages to a particular application need not be used for all applications. Also, not all limitations need be implemented in methods, systems and/or apparatus including one or more concepts of the present invention.

It is noted that the foregoing has outlined some of the more pertinent objects and embodiments of the present invention. This invention may be used for many applications. Thus, although the description is made for particular arrangements and methods, the intent and concept of the invention is suitable and applicable to other arrangements and applications. It will be clear to those skilled in the art that modifications to the disclosed embodiments can be effected without departing from the spirit and scope of the invention. The described embodiments ought to be construed to be merely illustrative of some of the more prominent features and applications of the invention. Other beneficial results can be realized by applying the disclosed invention in a different manner or modifying the invention in ways known to those familiar with the art.

The word “comprising”, “comprise”, or “comprises” as used herein should not be viewed as excluding additional elements. The singular article “a” or “an” as used herein should not be viewed as excluding a plurality of elements. Unless the word “or” is expressly limited to mean only a single item exclusive from other items in reference to a list of at least two items, then the use of “or” in such a list is to be interpreted as including (a) any single item in the list, (b) all of the items in the list, or (c) any combination of the items in the list. Ordinal terms in the claims, such as “first” and “second” are used for distinguishing elements and do not necessarily imply order of operation.

The flowchart and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The use of variable names in describing operations in a computer or algorithm does not preclude the use of other variable names for achieving the same function.

What is claimed is:

1. A computer method comprising running operations on at least one data processing device, the operations comprising:
maintaining at least one database of information embodied on a medium including historical realization profiles relating to a set of past courses of events;
creating at least one similarity rubric responsive to the historical realization profiles; and
deriving a predicted realization profile for a new course of events responsive to partial knowledge of a new instance and responsive to the similarity rubric.

2. The method of claim 1, wherein the rubric comprises a similarity manifold in an N-dimensional space, where N is a number of characteristics maintained for historical realization profile.

3. The method of claim 2, wherein creating a predicted realization profile comprises
projecting a point onto the manifold, the point being derived from features taken from the partial knowledge, to yield a projected point; and
reading out additional features relating to the new instance responsive to the projected point.

4. The method of claim 2, wherein the similarity manifold is derived responsive to the following equation:
$$\frac{\partial u}{\partial t} = \frac{{β}^{2}}{{σ}^{2}} ( - {I}_{C} u + ( 1 - {I}_{C} ) ( 1 - u ) ) + ɛ Δ u + {Δ}^{-} u$$
where,
u is a smooth function to be computed, corresponding to the functional representation of the desired manifold. As already mentioned, the value of u far from the manifold is 1, and tends to 0 as one approaches manifold.
C is the initial cloud of points.
I is an indicator function of C, i.e. I_{C}(C)={1} and I_{C}(Rn/C)={1},
Δ⁻u is the negative part of the Laplacian of u, and
σ is the finest possible scale (determined by the resolution of the grid, e.g. for most common σ=1, two distinguishable points are two grid nodes.

5. The method of claim 4, wherein
β=ln(7+sqrt(48)) and
ε<<1.

6. The method of claim 1, wherein creating a similarity rubric comprises:
defining a similarity measure for the historical realization profiles;
applying clustering analysis to segment the historical realization profiles into groups, each group having a respective representative profile;
choosing one of the groups as relating to the new instance; and
taking, as a predicted realization profile for the new instance, the respective representative profile for the group resulting from the predicting.

7. The method of claim 1, wherein
creating a similarity rubric comprises:
representing past data in matrix form; and
factoring the matrix to create a lower rank approximation; and
deriving comprises selecting a vector from the lower rank approximation as the predicted realization profile.

8. An event management method comprising
the method of claim 1 and
committing resources to an actual course of action responsive to the predicted realization profile.

9. A system comprising:
at least one medium for embodying machine readable data and program code;
at least one interface for communicating externally;
at least one processor adapted to run operations responsive to the medium and interface, the operations comprising
maintaining at least one database of information embodied on the medium and including historical realization profiles relating to a set of past courses of events;
creating at least one similarity rubric responsive to the historical realization profiles; and
deriving a predicted realization profile for a new course of events responsive to partial knowledge of a new instance and responsive to the similarity rubric.

10. The system of claim 9, wherein the rubric comprises a similarity manifold in an N-dimensional space, where N is a number of characteristics maintained for historical realization profile.

11. The system of claim 9, wherein creating a predicted realization profile comprises projecting features of the partial knowledge onto the manifold.

12. The system of claim 9, wherein creating a similarity rubric comprises:
defining a similarity measure for the historical realization profiles;
applying clustering analysis to segment the historical realization profiles into groups, each group having a respective representative profile;
predicting which group the new instance falls into;
using the respective representative profile as a predicted realization profile for the new instance.

13. The system of claim 9, wherein
creating a similarity rubric comprises:
representing past data in matrix form;
factoring the matrix to create a lower rank approximation;
deriving comprises selecting a vector from the lower rank approximation as the predicted realization profile.

14. An event management system comprising the system of claim 9 wherein the interface is adapted to facilitate committing resources to an actual course of action responsive to the predicted realization profile.

15. A computer program product for performing operations the computer program product comprising a storage medium readable by a processing circuit and storing instructions run by the processing circuit for performing a method comprising, the operations comprising:
maintaining at least one database of information embodied on a medium including historical realization profiles relating to a set of past courses of events;
creating at least one similarity rubric responsive to the historical realization profiles; and
deriving a predicted realization profile for a new course of events responsive to partial knowledge of a new instance and responsive to the similarity rubric.

16. The program product of claim 15, wherein the rubric comprises a similarity manifold in an N-dimensional space, where N is a number of characteristics maintained for historical realization profile.

17. The program product of claim 15, wherein creating a predicted realization profile comprises projecting features of the partial knowledge onto the manifold.

18. The program product of claim 15, wherein creating a similarity rubric comprises:
defining a similarity measure for the historical realization profiles;
applying clustering analysis to segment the historical realization profiles into groups, each group having a respective representative profile;
predicting which group the new instance falls into;
using the respective representative profile as a predicted realization profile for the new instance.

19. The program product of claim 15, wherein
creating a similarity rubric comprises:
representing past data in matrix form;
factoring the matrix to create a lower rank approximation;
deriving comprises selecting a vector from the lower rank approximation as the predicted realization profile.

20. The program product of claim 15 adapted to facilitate committing resources to an actual course of action responsive to the predicted realization profile.
